(19) United States
(12) Patent Application Publication
(10) Pub. No.: US 2007/0249695 A1
(43) Pub. Date: Oct. 25, 2007

(54) Preventive and/or Therapeutic Agents for Meniere's Disease

(76) Inventors: Kazuya Kawata, Saitama-shi (JP); Kenzo Nakao, Osaka-shi (JP); Ken Mizushima, Osaka-shi (JP); Shozo Matsuoka, Osaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/628,889

(22) PCT Filed: Jun. 29, 2005
(86) PCT No.: PCT/JP05/11905

§ 371 Date: Dec. 8, 2006

(30) Foreign Application Priority Data
Jun. 30, 2004 (JP) 2004-193921

(51) Int. Cl. A61K 31/4035; A61K 31/41; A61P 27/16

(52) U.S. Cl. 514/382; 514/416

(57) ABSTRACT

The present invention relates to preventive and/or therapeutic agents for Meniere's disease, which comprise a leukotriene antagonist (such as pranlukast hydrate) as an active ingredient. Leukotriene antagonists (such as pranlukast hydrate) are effective in ameliorating various symptoms, such as hearing impairment, tinnitus, a feeling of fullness in the ear and vertigo, thus being useful as a preventive and/or therapeutic agent for Meniere's disease.

### TECHNICAL FIELD

The present invention is concerned with preventive and/or therapeutic agents for Meniere's disease which comprise a leukotriene antagonist as an active ingredient.

### BACKGROUND ART

Meniere's disease is a disease characterized by the cardinal symptom of recurrent attacks of vertigo, which attacks are associated with the cochlear symptoms, such as hearing impairment, tinnitus and a feeling of fullness in the ear, etc.

The symptoms of vertigo occur in a wide variety of different conditions ranging from severe rotary vertigo to swaying or shaking immobile vertigo: one of such symptoms disappears within several minutes or continues for some hours, whereas the other involves repetition of ameliorated and aggravated dizziness over the extended period of time as long as several days. On the other hand, also known is cochlear Meniere's disease, which is associated with repeated appearance or manifestation and disappearance of hearing impairment, tinnitus and a feeling of fullness in the ear but without accompaniment of vertigo, and there is recently noticed a peculiar tendency for the disease to increase. The pathology of Meniere's disease is considered to refer to the physiological condition (endolymphatic hydrops) in which the lymphatic fluid accumulates excessively in the semicircular ducts and cochleae of the inner ear and although several theories or hypotheses, such as, immune abnormality have been proposed as the cause or etiology of the disease, no decisive or positive one has been established so far. The attacks are often complicated subsequently by autonomic nerve symptoms, such as nausea, vomiting, shoulder stiffness, headache and the like. Meniere's disease undergoes exacerbation in the course of recurrence of the attacks over a prolonged period of time, and when the condition of disease or the pathology progresses to the terminal stage, among others, hearing impairment gets worse to such a profound degree as may cause inconvenience and troubles to patients' daily lives, resulting eventually in development into the medium to advanced degree of hearing loss. Therefore, Meniere's disease has been designated as an intractable disease in Japan.

In addition, acute low-tone sensorineural hearing loss is the recently often diagnosed and identified disease, for which the pathology of endolymphatic hydrops is suggested, just as is the case with Meniere's disease, and is also called subtype Meniere's disease, since in some afflicted cases, the disease in fact has been transformed to Meniere's disease (refer to Nanbyoh to Zaitaku-Kea (Intractable Disease and Home Care), 7(12), p. 68-71, 2002).

As a drug of first choice against Meniere's disease, isosorbide, or an osmotic diuretic, is generally used in order to ameliorate or mitigate endolymphatic hydrops. However, it is known that isosorbide is not always adequately efficacious for hearing impairment tinnitus and a feeling of fullness in the ear, though it can act to control vertigo to some extent. In addition, isosorbide, being processed into an aqueous pharmaceutical preparation, is difficult to be carried by patients and causes inconvenience to them, while it tastes peculiar and also has to be dosed in larger amounts, thus being confronted with the problems, such as difficulties encountered in medication through ingestion. Consequently, there has been strongly demanded in clinical practice the development of a drug which elicits improved effect and facilitates medication to be attained.

In the meanwhile, 4-oxo-8-[4-(4-phenylbutoxy)benzoylamino]-2-(tetrazol-5-yl)-4H-1-benzopyran ½ hydrate (generic name: pranlukast hydrate), which is known as a leukotriene (LT) antagonist (refer to JP-A 61-050977), has practically been finding a clinical application as a therapeutic agent for asthma and allergic rhinitis, and has already been recognized as adequately safe. In contrast with this, the above-described pranlukast hydrate has not been known to be effective for Meniere's disease at all.

It is to be noted that ibudilast, already known to be an amelioration drug for asthma and cerebrovascular disorder, has been found to ameliorate various symptoms manifested by Meniere's disease (refer to Jibi Rinshoh (Otorhinic Clinics), vol. 83(12), pp. 1873-1883 (1990)).

### DISCLOSURE OF THE INVENTION

### Problem to Be Solved by the Invention

Therefore, the object of the present invention is to provide a preventive and/or therapeutic agent for Meniere's disease which elicits superior efficacy and facilitates medication to be attained.

### Means to Solve the Problem

The present inventors, with a specific view to solving the above-described problems, conducted intensive investigation, and as a result, not only found that pranlukast hydrate surprisingly shows ameliorating activity for various symptoms, such as vertigo, hearing impairment, tinnitus and a feeling of fullness in the ear, etc., but also discovered for the first time, as backed up with the clinical evidence, that the drug is effective as a preventive and/or therapeutic agent for Meniere's disease. Additionally, the inventors also found that pranlukast hydrate can produce excellent ameliorating effect for acute low-tone sensorineural hearing loss, or subtype Meniere's disease, and have completed the present invention.

Namely, the present invention relates to:
[1] A preventive and/or therapeutic agent for Meniere's disease which comprises a leukotriene antagonist,
[2] The preventive and/or therapeutic agent as described above under the item [1], wherein Meniere's disease is cochlear Meniere's disease,
[3] The preventive and/or therapeutic agent as described above under the item [1], wherein the leukotriene antagonist is pranlukast hydrate,
[4] The preventive and/or therapeutic agent as described above under the item [1], wherein the leukotriene antagonist is montelukast sodium or zafirlukast,
[5] The preventive and/or therapeutic agent as described above under the item [1], wherein said agent is an ameliorating agent for one or more symptoms selected from hearing impairment, tinnitus, a feeling of fullness in the ear and vertigo,
[6] The preventive and/or therapeutic agent as described above under the item [5], wherein the hearing impairment is acute low-tone sensorineural hearing loss,
[7] The preventive and/or therapeutic agent as described above under the item [3], wherein the daily dose of pranlukast hydrate is in the range of from 112.5 mg to 450 mg,
[8] The preventive and/or therapeutic agent as described above under the item [7], wherein the daily dose is 112.5 mg, 225 mg or 450 mg,
[9] The preventive and/or therapeutic agent as described above under the item [7], characterized in that said agent is administered in the dose of 225 mg each time, twice a day,
[10] The preventive and/or therapeutic agent as described above under the item [9], characterized in that said agent as processed into capsules each containing 112.5 mg is administered in the dose of two capsules each time, twice a day,
[11] A medical drug which comprises a combination of the preventive and/or therapeutic agent for Meniere's disease as described above under the item [1] with one kind or more kinds selected from diuretic drugs, antidinic drugs, ameliorating drugs for circulation in the inner ear, vitamins, antianxiety drugs, antiemetic drugs, antiallergic drugs and antihistamine drugs,
[12] A preventive and/or therapeutic agent for Meniere's disease which comprises pranlukast hydrate,
[13] The preventive and/or therapeutic agent as described above under the item [12], wherein the daily dose of pranlukast hydrate is 225 mg or 450 mg and said agent is the ameliorating agent for one or more symptoms selected from hearing impairment, tinnutis, a feeling of fullness in the ear and vertigo,
[14] A method for prevention and/or treatment of Meniere's disease, characterized in that said method comprises administering an effective dose of a leukotriene antagonist to a mammal,
[15] A use of a leukotriene antagonist in preparing a preventive and/or therapeutic agent for Meniere's disease,
[16] An ameliorating agent for one or more symptoms selected from hearing impairment, tinnitus, a feeling of fullness in the ear and vertigo, which comprises pranlukast hydrate, and
[17] The ameliorating agent as described above under the item [16] wherein the hearing impairment is acute low-tone sensorineural hearing loss.

The leukotriene (hereinafter referred to briefly as “LT”) antagonist of the present invention is understood to denote any compounds that elicit the LT antagonistic action as their principal action, and includes, for example, pranlukast hydrate, montelukast sodium, zafirlukast, MK-571, LY-203647, WY-46016, WY-48422, WY-49353, WY-49451, RG-12553, MDL-43291, CGP-44044A, RG-14524, LY-287192, LY-290324, L-695499, RPR-105735B, WAY-125007, OT-4003, LM-1376, LY-290154, SR-2566, L-740515, LM-1453, CP-195494, LM-1484, CR-3465, ablukast, pobilukast, sulukast, L-648051, RG-12525, RG-7152, SK&F-106203, SR-2640, WY-50295, iralukast sodium, verlukast, MCC-847, BAY-x-7195, ritolukast, cinalukast, CGP-44826, FK-011, YM-158, MEN-91507, KCA-757, RS-601, RS-635, S-36496, ZD-3523, DS-4574, pirodomast, AS-35, YM-57158, MCI826, NZ-107, 4414-CERM, YM-16638, Wy-48252, Wy-44329, Wy-48090,VUF-4679, tomelukast, SM-11044, SC-39070, OT-3473, N-2401, LY-243364, L-649923, doqualast, DP-1934, YM-17751, Wy-47120, VUF-K-8707, SK&F-88046, SK&F-101132, SK&F-102922, LY-137617, LY-163443, LY-302905, L-647438, L-708738, KY-234, FPL-55712, CP-288886, S-36527, CS-615, MDL-19301D, SCH-40120, ZD-3705 and so on. In addition, the LT antagonist of the present invention include not only the ones which have already been found out in the past up to now but also the ones which will be found in the future from now onward. The LT antagonist preferably includes pranlukast hydrate, montelukast sodium and zafirlukast, and more preferably pranlukast hydrate.

Pranlukast hydrate used in the present invention is 4-oxo-8-[4-(4-phenylbutoxy)benzoylamino]-2-(tetrazol-5-yl)-4H-1-b enzopyran ½ hydrate represented by the formula (A): 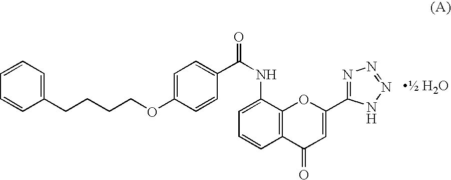
[Method for Preparing the Compound of the Present Invention]

Preparation of pranlukast hydrate can be preformed in accordance with the method described in JP-A S61-050977. The other LT antagonists can also be prepared by the known methods.

[Toxicity]

Pranlukast hydrate exhibits a very low degree of toxicity and has been recognized as safe enough to be used as a medical drug. Referring to the acute toxicity, for example, pranlukast hydrate shows the minimum lethal dose of not less than 2000 mg/kg, when given to mice and rats (both male and female) orally or subcutaneously.

[Application to Medical Drugs]

Pranlukast hydrate produces the ameliorating effect for various symptoms, such as hearing impairment (e.g. sensorineural hearing loss (e.g. acute low-tone sensorineural hearing loss, etc.), etc.), tinnitus, a feeling of fullness in the ear and vertigo, etc., and is useful as a preventive and/or therapeutic agent for Meniere's disease.

Delayed endolymphatic hydrops is also known as a disease in which sudden hearing impairment or sensorineural hearing loss precedes the onset or manifestation of the symptoms similar to those of Meniere's disease by several years to not less than 10 years. Pranlukast hydrate, which elicits the ameliorating action against Meniere's disease, is consequently considered to be also useful as a preventive and/or therapeutic agent for delayed endolymphatic hydrops.

In the present invention, prevention of Meniere's disease includes the suppression of onset or manifestation of various symptoms manifested by Meniere's disease.

The preventive and/or therapeutic agent of the present invention may be administered as a concomitant drug agent or preparation produced by combination thereof with other drugs for the purposes of 1) complementation or supplementation and/or reinforcement of the preventing and/or treating effect produced by the active ingredient, 2) amelioration or improvement of pharmacokinetic characteristics/absorption and reduction of doses of the active ingredient and/or 3) alleviation of side effects or adverse reactions of the active ingredient.

The concomitant drug agents or preparations of the LT antagonists with other drugs may be administered in the form of the combination drug agents or preparations having both of these two components incorporated in one pharmaceutical preparation or may be administered in separate pharmaceutical preparations. Administration in the form of separate pharmaceutical preparations includes the simultaneous and time-lag administrations. In the latter case, the LT antagonist may be administered first in advance, whereafter the other drugs may be applied, and vice versa, and the administration methods may be the same or different.

The other drugs may be either low-molecular compounds or macromolecular proteins, polypeptides, polynucleotides (DNAs, RNAs, and genes), antisense RNAs, decoys, antibodies or vaccines and so on. The dose of the other drugs can be appropriately selected, while taking their clinically applied doses as a criterion. Additionally, the formulation ratio of the leukotriene antagonist and the other drugs can be suitably selected, depending upon the age and body weight of a subject to be administered, the method and time of administration the disease to be treated, the symptom, the drug combination, etc. For example, the other drugs may be used at ratios of from 0.01 to 100 parts by weight per part by weight of pranlukast hydrate. The other drugs may be administered by combination in appropriate proportions with one or more kinds being arbitrarily selected from the below-described same or different groups.

Said other drugs include, for example, diuretic drugs, antidinic drugs, ameliorating drugs for circulation in inner ear, vitamin drugs, antianxiety drugs, antiemetic drugs, antiallergic drugs and antihistamine drugs and so on.

The diuretic drugs include, for example, isosorbide, acetazolamide, furosemide and so on. The antidinic drugs include, for example, sodium hydrogen carbonate solution, betahistine mesylate, diphenhydramine salicylate, difenidol hydrochloride, chlorpromazine hydrochloride, perphenazine and so on. The ameliorating drugs for circulation in the inner ear include, for example, betahistine mesylate, disodium adenosine triphosphate, kallidinogenase and so on. The vitamins include, for example, mecobalamin and so on. The antianxiety drugs include, for example, chlordiazepoxide, oxazolam, diazepam, bromazepam, ethyl loflazepate, alprazolam, lorazepam and soon. The antiemetic drugs include, for example, metoclopramide hydrochloride, domperidone, perphenazine maleate and so on. The antiallergic drugs include, for example, sodium cromoglycate, tranilast, amlexanox, repirinast, ibudilast., pemirolast potassium, tazanolast, nedocromil, cromoglicate, israpafant, ketotifen fumarate, azelastine hydrochloride, oxatomide, mequitazine, terfenadine, emedastine fumarate, epinastine hydrochloride, ebastine, cetirizine hydrochloride, olopatadine hydrochloride, loratadine, fexofenadine, ozagrel hydrochloride, imitrodast sodium, seratrodast, ramatroban, domitroban calcium hydrate, KT-2-962, suplatast tosylate and so on. The antihistaminc drugs include, for example, dimenhydrinate and so on.

In addition, the other drugs include not only the ones which have been found out in the past up to now on the basis on the above-mentioned mechanism but also the ones which will be discovered in the future from now onward.

In order to use the LT antagonists or the concomitant drug agents or preparations (the combination drug agents or preparations) comprising the LT antagonist in combination with the other drugs for the above-mentioned purposes, they are normally administered systemically or topically in the oral or parenteral dosage form.

For example, the dose of planlukast hydrate varies depending upon the age and body weight of a patient, and the symptom, therapeutic effect, method of administration, treatment time and so on, and the administration may be effected individually and particularly so as to obtain the desired effect of the present invention. Its daily dose for adults, for example, preferably ranges from about 25 mg to about 2500 mg, more preferably from about 112.5 mg to about 450 mg, further more preferably from about 225 mg to about 450 mg.

The method of administration of pranlukast hydrate is preferably the oral route of administration. Referring to the frequency of administration, such drug is administered once to several times a day, preferably once to four times a day, more preferably once to twice a day. In addition, one to several capsules may be administered orally each time, while one to two capsules are preferably applied orally, and two capsules each containing 112.5 mg of pranlukast hydrate is most preferably administered each time, twice a day.

Because the dose, the method and frequency of administration fluctuate according to a variety of different conditions, naturally, it may in some instances suffice that they fall short of, or exceed, the above-mentioned ranges.

In the case of administration of the LT antagonist or the concomitant drug agent or preparation comprising the LT antagonist and other drugs, they are utilized in the dosage forms, such as solid and liquid pharmaceutical preparations for oral use, which are intended for oral administration, or injectable solutions or pharmaceutical preparations, topical. pharmaceutical preparations for external application, suppositories, ophthalmic solutions, inhalants, etc. , which are intended for parenteral administration.

As the solid pharmaceutical preparations for oral use intended for oral administration, for example, there may be mentioned tablets, pills, capsules, powders and granules. The capsules include hard and soft capsules.

In such solid pharmaceutical preparations for oral use, one or more active substances are mixed or granulated (e.g., through agitation granulation, fluidized bed granulation, dry granulation, tumbling agitation fluidized bed granulation, etc.) as such or in conjunction with additives, and then processed into pharmaceutical preparations (e.g., encapsulation, tablet compression, etc.) in accordance with the conventional processes. Additives in one or more kinds may appropriately be formulated. The additives include, for example, excipients (e.g., lactose, mannitol, glucose, microcrystalline cellulose, corn starch, etc.), binders (e.g., hydroxypropyl cellulose, polyvinylpyrrolidones, magnesium metasilicoaluminate, etc.), dispersants (e.g., corn starch, etc.), disintegrators (e.g., calcium fibrinoglycolate, etc.), lubricants (e.g., magnesium stearate, etc.), stabilizers, solubilizing agents (e.g., glutamic acid, aspartic acid, etc.), water-soluble polymers (e.g., celluloses (e.g., hydroxypropyl cellulose, hydroxypropyl methylcellulose, methylcellulose, etc.), synthetic polymers (e.g., polyehtylenegrylcols, polyvinylpyrrolidones, polyvinyl alcohols, etc.) etc.), and sweeteners (e.g., white sugar, powdered sucrose, sucrose, fructose, glucose, lactose, hydrogenated maltose starch syrup, powdered hydrogenated maltose starch syrup, glucose fructose syrup, honey, sorbitol, martitol, mannitol, xylitol, erythritol, aspartame, saccharin, saccharin sodium, etc.). The solid pharmaceutical preparations may be covered with a coating agent (e.g., white sugar, gelatin, hydroxypropyl cellulose, hydroxypropyl methylcellulose phthalate, etc.), as the case may be, or covered with two or more layers. Furthermore, the solid pharmaceutical preparations include the capsules composed of absorbable materials, such as gelatin.

Examples of the liquid pharmaceutical preparations for oral use intended oral administration include pharmaceutically allowable aqueous solutions, suspensions and emulsions, syrups, pharmaceutical preparations to be dissolved on the occasion of use (e.g., dry syrups) and elixirs, etc. In such liquid pharmaceutical preparations, one or more active substances are dissolved, suspended or emulsified in a commonly used diluent (e.g., purified water, ethanol, or mixtures thereof, etc.). Furthermore, such liquid pharmaceutical preparations may contain wetting agents, suspending agents, emulsifiers, sweetening agents, flavors, fragrances, preservatives, buffers, etc. In addition, the liquid pharmaceutical preparations for oral use include the pharmaceutical preparations to be dissolved on the occasion of use (e.g., dry syrups) which permit the above-described solid pharmaceutical preparations for oral use to be taken in the form of a solution.

Out of the pharmaceutical preparations intended for parenteral administration, topical pharmaceutical preparations for external application include, for example, ointments, gels, creams, cataplasms, pastes, liniments, nebulas, inhalants, sprays, eye drops or ophthalmic solutions, collunaria and the like. These pharmaceutical preparations each contain one or more active substances and are prepared by any known processes or in accordance with the commonly used formulations.

The ointments are prepared in accordance with the known or commonly used formulations. For example, one or more active substances are triturated or molten in a base to produce and prepare such an ointment. The ointment base is selected from the known or commonly used bases. For example, use is made of admixtures of one or more kinds being selected from higher fatty acids or fatty acid esters(e.g., adipic acid, myristic acid, palmitic acid, stearic acid, oleic acid, adipic acid esters, myristic acid esters, palmitic acid esters, stearic acid esters, oleic acid esters, etc.), waxes (e.g., beeswax, whale wax, ceresin, etc.), surface active agents (e.g., polyoxyethylene alkyl ether phosphoric acid esters, etc.), higher alcohols (e.g., cetanol, stearyl alcohol, setostearyl alcohol, etc.), silicon oils (e.g., dimethyl polysiloxane, etc.), hydrocarbons (e.g., hydrophilic petrolatum, white petrolatum, purified lanolin, liquidparaffin, etc.), glycols (e.g., ethyleneglycol, diethylene glycol, propylene glycol, polyethylene glycols, macrogol, etc.), vegetable oils (e.g., castor oil, olive oil, sesame oil, turpentine oil, etc.), animal oils (e.g., mink oil, egg yolk oil, squalane, squalene, etc.), water, absorption accelerators and skin-rash preventives. The ointments may further contain humectants, preservatives, stabilizers, antioxidants, perfumes, etc.

The gels are prepared in accordance with the known or commonly used formulations. For example, one or more active substances are dissolved in a base to produce and prepare such a gel. The gel bases are selected from known or commonly used bases. For example, use is made of admixtures of one or more kinds being selected from lower alcohols (e.g., ethanol, isopropyl alcohol, etc.), gelling agents (e.g., carboxymethyl cellulose, hydroxyethyl cellulose, hydroxypropyl cellulose, ethyl cellulose, etc.), neutralizing agents (e.g., triethanolamine, diisopropanolamine, etc.), surface active agents (e.g., polyethylene glycol monostearate, etc.), gums, water, absorption accelerators, and skin-rash preventives. The gels may further contain preservatives, antioxidants, perfumes, etc.

The creams are prepared in accordance with the known or commonly used formulations. For example, one or more active substances are molten or emulsified in a base to produce and prepare such a cream. The cream bases are selected from the known or commonly used bases. For example, use is made of admixtures of one or more kinds being selected from higher aliphatic acid esters, lower alcohols, hydrocarbons, polyhydric alcohols (e.g., propylene glycol, 1,3-butylene glycol, etc.), higher alcohols (e.g., 2-hexyl decanol, cetanol, etc.), emulsifiers (e.g., polyoxyethylene alkyl ethers, aliphatic acid esters, etc.), water, absorption accelerators, and skin-rash preventives. The creams may further contain preservatives, antioxidants, perfumes, etc.

The cataplasms are prepared in accordance with the known or commonly used formulations. For example, one or more active substances are molten in abase, followed by application through spreading of the kneaded material over over a supporting material to prepare such a cataplasm. The cataplasms are selected from the known or commonly used bases. For example, use is made of admixtures of one or more kinds being selected from thickening agents (e.g., polyacrylic acids, polyvinylpyrrolidones, gum arabic, starches, gelatin, methylcellulose, etc.), wetting agents (e.g., urea, glycerol, propylene glycol, etc.), fillers (e.g., kaolin, zinc oxide, talc, calcium, magnesium, etc.), water, solubilizing agents, tackifiers, and skin-rash preventives. The cataplasms may further contain preservatives, antioxidants, perfumes, etc.

The pastes are prepared in accordance with the known or commonly used formulations. For example, one or more active substances are molten in a base, followed by application through spreading over a supporting material to prepare such a paste. The paste base is selected from the known or commonly used bases. For example, use is made of admixtures of one or more kinds being selected from polymer bases, fats and oils, higher fatty acids, tackifiers and skin-rash preventives. The pastes may further contain preservatives, antioxidants, perfumes, etc.

The liniments are prepared in accordance with the known or commonly used formulations. For example, one or more active substances are dissolved, suspended or emulsified in one or more kinds being selected from water, alcohols (e.g., ethanol, polyethylene glycols, etc.), higher fatty acids, glycerol, soaps, emulsifiers, suspending agents, etc. The liniments may further contain preservatives, antioxidants, perfumes, etc.

The nebulas, inhalants and sprays may contain the commonly used diluents, as well as stabilizers, such as sodium hydrogensulfite and buffers capable of providing isotonicity, such as isotonic agents (e.g., sodium chloride, sodium citrate, or citric acid, etc.). The process for preparation of sprays is described in detail, for example, in the U.S. Pat. Nos. 2,868,691 and 3,095,355. In addition, the sprays can be made into aerosols.

The injectable pharmaceutical preparations intended for parenteral administration include solutions, suspensions, emulsions and solid injectable pharmaceutical preparations to be dissolved or suspended in a solvent on the occasion of use. The injectable pharmaceutical preparations are prepared by dissolving, suspending or emulsifying one or more active substances in a solvent. As such a solvent, for example, there may be used distilled water for injection, physiological saline, vegetable oils, alcohols such as propylene glycol, polyethylene glycols and ethanol, etc., and combinations thereof. Such injectable pharmaceutical preparations may further contain stabilizers, solubilizing agents (e.g., glutamic acid, aspartic acid, Polysolvate 80 (the registered trademark), etc.), suspending agents, emulsifiers, pain-soothing agents, buffers, preservatives, etc. The injectable pharmaceutical preparations are sterilized at the final step or prepared by the aseptic manipulation process. Alternatively, sterile solid pharmaceutical preparations, for example, lyophilizates, are produced and used by dissolving them in sterile distilled water for injection or other solvents prior to their use.

The inhalants for parenteral administration may be in the form of aerosols, powders for inhalation or liquid pharmaceutical preparations for inhalation. The liquid pharmaceutical preparations for inhalation may be in such a form as may be used by dissolving or suspending in water or other proper media. These inhalants are prepared by the known processes.

For example, the liquid pharmaceutical preparations for inhalation are prepared by selecting appropriately preservatives (e.g., benzalkonium chloride, parabens, etc.), colorants, buffering agents (e.g., sodium phosphate, sodium acetate, etc.), isotonic agents (e.g., sodium chloride, concentrated glycerol, etc.), thickening agents (e.g., carboxyvinyl polymers, etc.), absorption accelerators, etc., as the case may be.

The powders for inhalation are prepared by selecting appropriately lubricants (e.g., stearic acid and salts thereof, etc.), binders (e.g., starches, dextrin, etc.), excipients (e.g., lactose, cellulose, etc.), colorants, preservatives (e.g., benzalkonium chloride, parabens, etc.), absorption accelerators, etc., if necessary.

In the case of administration of the liquid pharmaceutical preparations for inhalation, a sprayer (e.g., atomizer, nebulizer, etc.) is normally used. On the occasion of administration of the powders for inhalation, a powder inhaler is normally used.

Other compositions intended for parenteral administration include the suppositories for rectal application and the pessaries for vaginal application as formulated by the ordinary methods, both of which contain one or more active substances.

### Effect of the Invention

The present invention can provide the effective preventive and/or therapeutic agents for Meniere's disease.

### Best Mode for Carrying out the Invention

The present invention is described below in detail by referring to Examples (clinical pharmacological test), but the present invention is not understood to be limited thereto.

### EXAMPLE 1

Pranlukast hydrate (trade name: onon capsule) was administered to 19 patients afflicted with Meniere's disease at a daily dose of 450 mg (two capsules each containing 112.5 mg individually after breakfast and after dinner) for the period of not less than three weeks (up to 24 weeks max.) to test and determine the efficacy. The overall efficacy (therapeutic) assessment was performed mainly on the basis of the results of nystagmus and hearing tests, while also taking into consideration the subjective symptoms (rotary vertigo, tinnitus, a feeling of fullness in ear, hearing rimpairment, etc.) and the extent or degree of hearing impairment (average hearing, method of quartering). In consequence of this, the efficacy was recognized and determined in 17(89.5%) out of 19 patients afflicted with Meniere's disease. The breakdown of efficacy indicated that the drug agent elicites the efficacy in 15 out of 19 patients complaining of the subjective symptoms (78.9%) and in 15 out of 19 patients suffering from hearing impairment (78.9%), while the drug agent shows 100% (effective in 6 patients out of 6 patients) of the effective rate in the patients suffering from vertigo (rotary vertigo or stagger).

The above-described results suggested that pranlukast hydrate, when administered to the patients afflicted with Meniere's disease, can relieve and ameliorate vertigo and additionally can produce the ameliorating effect against hearing impairment, tinnitus and a feeling of fullness in the ear against which the existing drugs (diuretic drug, isosorbide) have hardly been found to exhibit the efficacy. These results demonstrated that pranlukast hydrate is effective as a therapeutic agent against Meniere's disease.

### EXAMPLE 2

Pranlukast hydrate (trade name: Onon capsule) was administered to the patients suffering from acute low-tone sensorineural hearing loss concurrently with a feeling of fullness in the left ear at a daily dose of 450 mg (two capsules each containing 112. 5 mg given individually after breakfast and dinner) for two months to investigate into the efficacy against hearing impairment, the major complaint. In connection to the above test, it is to be noted that selection of patients was performed in accordance with a draft of the Criteria for Diagnosis proposed by Study Group for Acute Severe Hearing Loss of the Ministry of Health, Labor and Welfare of Japan (refer to Audiology Japan., 45, 161-166 (2002)).

The efficacy evaluation was performed on the basis of the Criteria for Efficacy Evaluation drawn up by Study Group for Acute Severe Hearing Loss of the Ministry of Health, Labor and Welfare of Japan (refer to Audiology Japan., 45, 161-166 (2002)); the cases, whose hearing levels at the three frequencies (125, 250, and 500 Hz) in the lone-tone range were restored to a level of less than 20 dB or were restored to the same level as the ones in the unaffected, sound ears, were classified as “healed”, and the cases, whose average hearing levels at the three frequencies in the low-tone range were recovered by not less than 10 dB but failed to heal, were classified as “ameliorated”, while the cases, whose average hearing levels at the three frequencies in the low-tone range were recovered by less than 10 dB, were classified as “unchanged”, with the remaining cases other than the above-mentioned cases being classified as “worsened”.

The test results revealed that pranlukast hydrate displays a tendency of amelioration both in the subjective symptoms and hearing within one week of administration, and subsequently acts to exhibit amelioration in the average hearing levels (average hearing level recovered by 19 dB) after one month of administration, as shown in Table 1, and furthermore functions to allow the hearing to be restored to a level similar to the one in the unaffected, sound ear after two months of administration, thereby leading to healing.

TABLE 1

| | Hearing in the affected ear | | | | Hearing in the sound ear | | | |
|---|---|---|---|---|---|---|---|---|
| | | | | Average | | | | Average |
| Administration | 125 Hz | 250 Hz | 500 Hz | hearing | 125 Hz | 250 Hz | 500 Hz | hearing |
| period | (dB) | (dB) | (dB) | level (dB) | (dB) | (dB) | (dB) | level (dB) |
| Before | 40 | 50 | 35 | 42 | 25 | 15 | 10 | 17 |
| administration | | | | | | | | |
| After one | 30 | 25 | 15 | 23 | 25 | 15 | 15 | 18 |
| month | | | | | | | | |
| After two | 25 | 20 | 10 | 18 | 25 | 15 | 10 | 17 |
| months | | | | | | | | |

The above results have demonstrated that pranlukast hydrate can elicit the hearing-ameliorating action in the patients afflicted with acute low-tone sensorineural hearing loss.

### Pharmaceutical0-Preparation Example

Pharmaceutical-Preparation Example 1: Preparation of capsules Pranlukast hydrate (40 kg), lactose (19 kg) and additives (appropriate amounts) were spray-drying granulated by the conventional procedure to give the granulated product containing pranlukast hydrate at a ratio of 625 mg per 1 g of the granulated product. The resultant granulated product was filled in No. 3 capsules in accordance with the conventional procedure to produce the capsules each having a pranlukast hydrate content of 112.5 mg.

### INDUSTRIAL APPLICABILITY

Pranlukast hydrate, which can produce a preventive and/or therapeutic effect against Meniere's disease, is very useful as an ameliorating agent for various symptoms of said disease (hearing impairment, tinnitus, a feeling of fullness in the ear and vertigo, etc.).

1. A preventive and/or therapeutic agent for Meniere's disease which comprises a leukotriene antagonist.

2. The preventive and/or therapeutic agent according to claim 1, wherein Meniere's disease is cochlear Meniere's disease.

3. The preventive and/or therapeutic agent according to claim 1, wherein the leukotriene antagonist is pranlukast hydrate.

4. The preventive and/or therapeutic agent according to claim 1, wherein the leukotriene antagonist is montelukast sodium or zafirlukast.

5. The preventive and/or therapeutic agent according to claim 1, wherein said agent is an ameliorating agent for one or more symptoms selected from hearing impairment, tinnitus, a feeling of fullness in the ear and vertigo.

6. The preventive and/or therapeutic agent according to claim 5, wherein hearing impairment is acute low-tone sensorineural hearing loss.

7. The preventive and/or therapeutic agent according to claim 3, wherein a daily dose of pranlukast hydrate is in the range of from 112.5 mg to 450 mg.

8. The preventive and/or therapeutic agent according to claim 7, wherein the daily dose is 112.5 mg, 225 mg or 450 mg.

9. The preventive and/or therapeutic agent according to claim 7, characterized in that said agent is administered to a patient in the dose of 225 mg each time twice a day.

10. The preventive and/or therapeutic agent according to claim 9, characterized in that said agent is administered to a patient in the dose of two capsules each containing 112.5 mg each time twice a day.

11. A medical drug which comprises a combination of the preventive and/or therapeutic agent for Meniere's disease according to claim 1 with one or more kinds selected from diuretic drugs, antidinic drugs, ameliorating drugs for circulation in the inner ear, vitamins, antianxiety drugs, antiemetic drugs, antiallergic drugs and antihistaminic drugs.

12. A preventive and/or therapeutic agent for Meniere's disease which comprises pranlukast hydrate.

13. The preventive and/or therapeutic agent according to claim 12, wherein a daily dose of pranlukast hydrate is 225 mg or 450 mg and said agent is the ameliorating agent for one or more symptoms selected from hearing impairment, tinnitus, a feeling of fullness in the ear and vertigo.

14. A method for prevention and/or treatment of Meniere's disease, characterized in that said method comprises administering an effective dose of a leukotriene antagonist to a mammal.

15. (canceled)

16. An ameliorating agent for one or more symptoms selected from hearing impairment, tinnitus, a feeling of fullness in the ear and vertigo, which comprises pranlukast hydrate.

17. The ameliorating agent according to claim 16, wherein hearing impairment is acute low-tone sensorineural hearing loss.
